(19) United States
(12) Patent Application Publication
(10) Pub. No.: US 2024/0398572 A1
(43) Pub. Date: Dec. 5, 2024

(54) Jaw Graft Reconstruction Assembly, System and Procedure

(71) Applicants: Baber Khatib, Portland, OR (US); Justin Lyons, Jacksonville, FL (US); Andrew Strickland, Jacksonville, FL (US); Scott Eibel, Jacksonville, FL (US); Matt Radloff, Jacksonville, FL (US)

(72) Inventors: Baber Khatib, Portland, OR (US); Justin Lyons, Jacksonville, FL (US); Andrew Strickland, Jacksonville, FL (US); Scott Eibel, Jacksonville, FL (US); Matt Radloff, Jacksonville, FL (US)

(21) Appl. No.: 18/734,952

(22) Filed: Jun. 5, 2024

Related U.S. Application Data
(60) Provisional application No. 63/471,238, filed on Jun. 5, 2023.

(51) Int. Cl. A61F 2/28; A61F 2/30; A61F 2/46; A61L 27/36

(52) U.S. Cl. CPC A61F 2/2875; A61L 27/3608; A61L 27/365; A61F 2002/2889; A61F 2002/30957; A61F 2002/30991; A61F 2002/4633; A61F 2002/4649

(57) ABSTRACT

A method for designing and producing a reconstruction assembly for implantation onto a maxilla or mandible to replace a resected or damaged portion, the method utilizing a virtual and a physical 3-D model of a patient, as well as the reconstruction assembly itself, the reconstruction assembly having a fixation implant, a dental prosthesis, a fibular flap, a removable alignment outrigger joined to the dental prosthesis, and implant posts.

### BACKGROUND OF THE INVENTION

This invention relates generally to facial reconstruction implant systems and procedures wherein 3-D virtual and/or physical models are used, such as for example in bone reconstruction, orthognathic surgery or related bone treatments involving osteotomies or bone repair, and in particular to jaw replacement surgery using patient-specific models, i.e., models customized and configured based on 3-dimensional computer-aided design to recreate in physical form a precise 3-dimensional copy of the maxilla or mandible of the patient. The invention also relates to the method of designing, manufacturing and utilizing such models in a surgical procedure wherein a portion of the maxilla or mandible is replaced by an assembly comprising a dental prosthesis, a fibula flap, implant posts, and a fixation implant, the model ensuring that the assembly is properly configured and oriented prior to implantation in the patient. The invention also relates to the assembly itself, wherein the dental prosthesis is joined to the fibula flap by the implant posts and the fibula plant is mounted to the fixation bone plate.

A maxilla or mandible may be malformed, diseased, fractured or otherwise damaged to the extent that it needs to be surgically repaired by removing a portion of the bone, orienting segments of fibula bone to recreate a reconstruction arch or flap, securing a dental prosthesis to the reconstruction flap, and securing the combined reconstruction flap and dental prosthesis to the patient's remaining maxilla or mandible segments. Modern surgery makes use of computer-aided design and manufacturing techniques whereby surgeons and technicians create virtual and/or physical 3-D models of a patient's bone structure in order to properly design, size, orient and configure reconstruction assembly. A representative procedure is discussed in detail in an article titled “Fibula Jaw in a Day: State of the Art in Maxillofacial Reconstruction” (J Oral Maxillfac Surg 74:1284.e1-1284.e15, 2016), the disclosure of which is incorporated herein by reference.

It is very important that the reconstructed mandible or maxilla reconstruction assembly be properly positioned and oriented relative to the patient's mandible or maxilla upon affixation in order to preserve the patient's previous or desired jaw articulation and junction between the opposing dental arches. It is an object of this invention to provide a novel method, system, procedure and reconstruction assembly that accomplishes this goal.

### BRIEF DESCRIPTION OF THE DRAWINGS

FIGS. 1 and 2 illustrate two views of a scanned maxilla of a patient showing the area to be surgically resected and preplaced with the reconstruction assembly.

FIG. 3 illustrates the planned resection of a fibula to create the bone segments for the construction of the fibula flap.

FIGS. 4 and 5 illustrate the desired positioning of the fibula flap and implant posts relative to the remaining maxilla on the 3-D scan of the patient.

FIG. 6 illustrates the implant post receiving bores in the dental prosthesis.

FIGS. 7 through 10 illustrate various views of an embodiment of the reconstruction assembly as positioned on a 3-D physical model of the patient's maxilla, wherein the dental prosthesis is properly positioned relative to the fibula by mechanically fastening the alignment outrigger to the fixation implant.

FIGS. 11 and 12 illustrate an alternative embodiment of a reconstruction assembly having peg projections extending from the alignment outrigger adapted to be seated in apertures in the fixation implant.

FIG. 13 illustrates the alternative embodiment of the reconstruction assembly of FIG. 11 or 12 as positioned on a 3-D physical model of the patient's maxilla, wherein the dental prosthesis is properly positioned relative to the fibula by peg projections extending from the alignment outrigger and seated in apertures in the fixation implant.

FIG. 14 illustrates an embodiment of a reconstruction assembly for a mandible mounted on an actual patient showing the dental prosthesis joined to the fibula flap and the fibula flap secured to the fixation implant prior to removal of the alignment outrigger.

### SUMMARY OF THE INVENTION

The invention is in general a process or procedure for modeling and producing a reconstruction assembly for implantation onto a maxilla or mandible (i.e., the upper or lower jaw, respectively) to replace a resected or damaged portion, as well as the reconstruction assembly itself. The use of 3-D modeling and implant design and development using computer assisted design is well known in the art. A representative reconstruction procedure utilizing 3-D modelling is discussed in detail in an article titled “Fibula Jaw in a Day: State of the Art in Maxillofacial Reconstruction” (J Oral Maxillfac Surg 74:1284.e1-1284.e15, 2016), the disclosure of which is incorporated herein by reference.

In brief, the jaw graft reconstruction method comprises the steps of:
(a) creating a virtual and a physical 3-D model of a patient's resected maxilla or mandible,
(b) designing and manufacturing components of a reconstruction assembly comprising a fixation implant contoured to the surface configuration of the patient's bone remaining after resection, a dental prosthesis, a removable alignment outrigger joined to the dental prosthesis, and implant posts,
(c) inserting the implant posts into a fibula,
(d) harvesting segments from the fibula,
(e) affixing the fixation implant to the resected 3-D physical model of the patient's maxilla or mandible,
(f) affixing the fibula segments to the fixation implant to create a fibula flap,
(g) mounting the alignment outrigger onto the fixation implant with the implant posts extending into bores in the dental prosthesis,
(h) applying resin to cement the dental prosthesis to the implant posts, whereby the alignment outrigger ensures the correct location and orientation of the dental prosthesis relative to the fixation implant,
(i) removing the alignment outrigger from the dental prosthesis and fixation implant, and
(j) transitioning the reconstruction assembly from the 3-D physical model onto the patient by securing the fixation implant onto the patient's bone.

The alignment outrigger and dental prosthesis are preferably manufactured as a single unit, with the alignment outrigger comprising one or more arms extending from the dental prosthesis and connected to a seating saddle adapted to mate with a portion of the fixation implant. The alignment outrigger is temporarily joined to the fixation implant by screws or pegs inserted into apertures provided in the fixation implant.

### DETAILED DESCRIPTION OF THE INVENTION

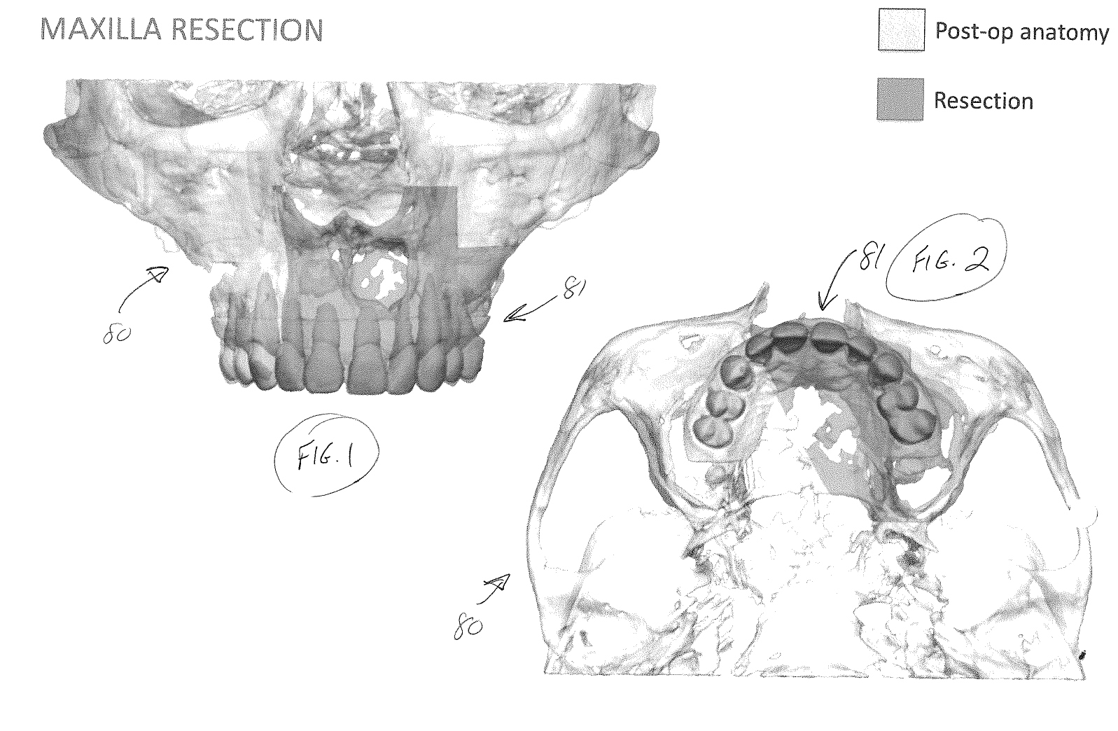

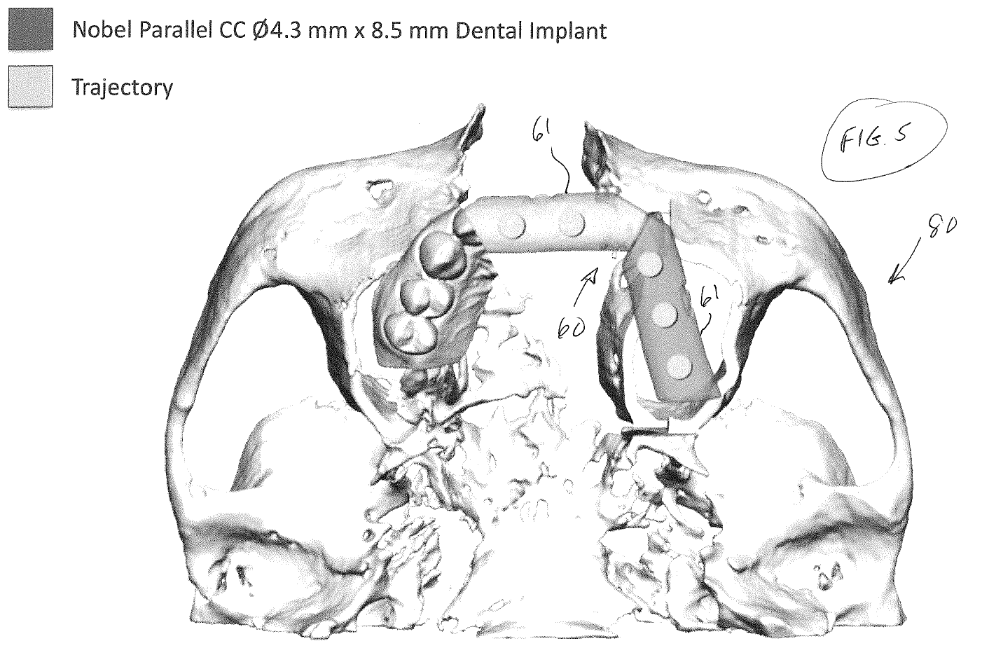

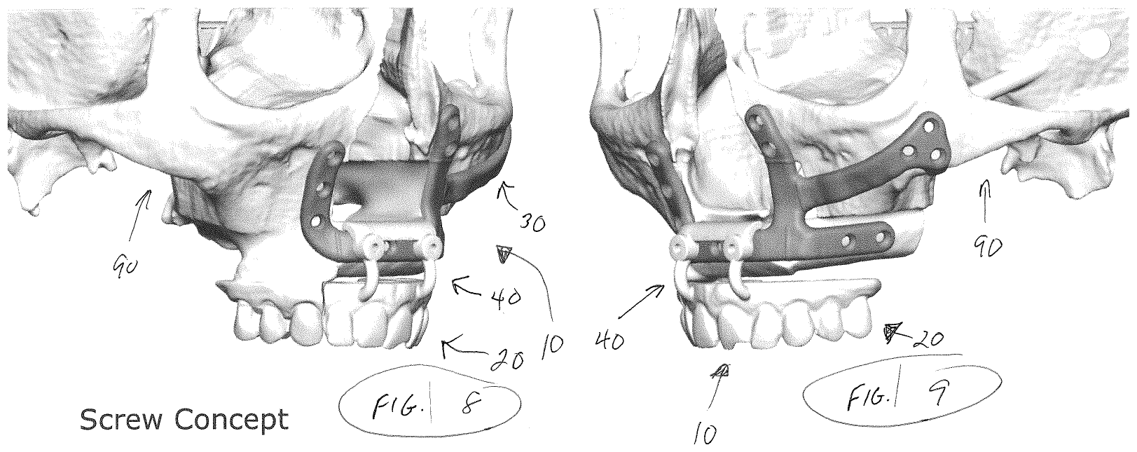

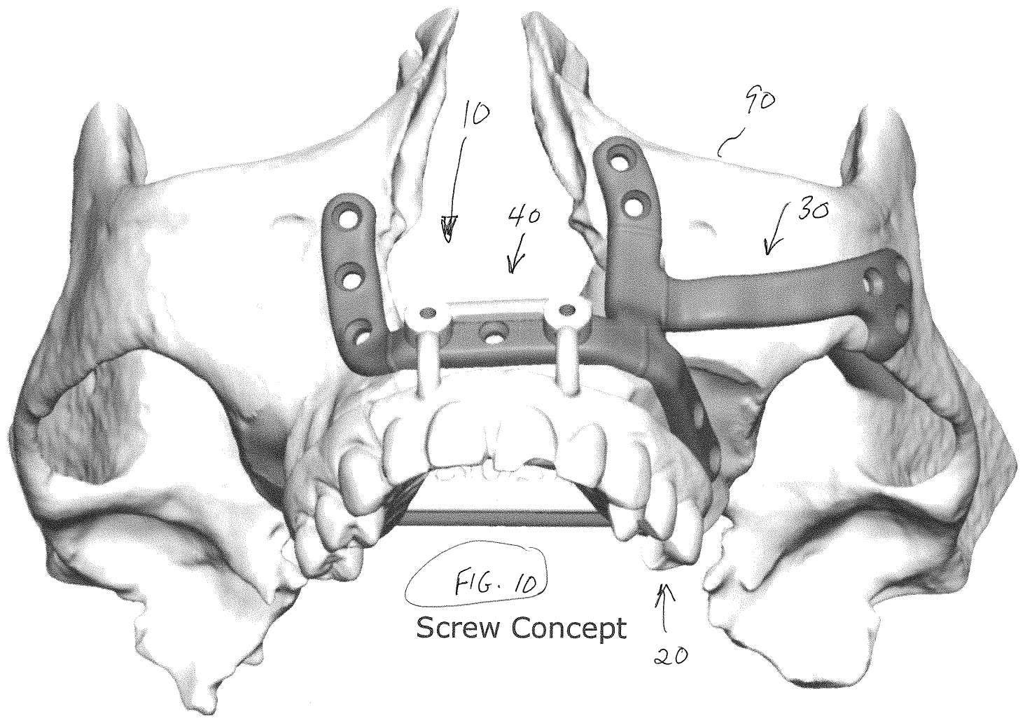

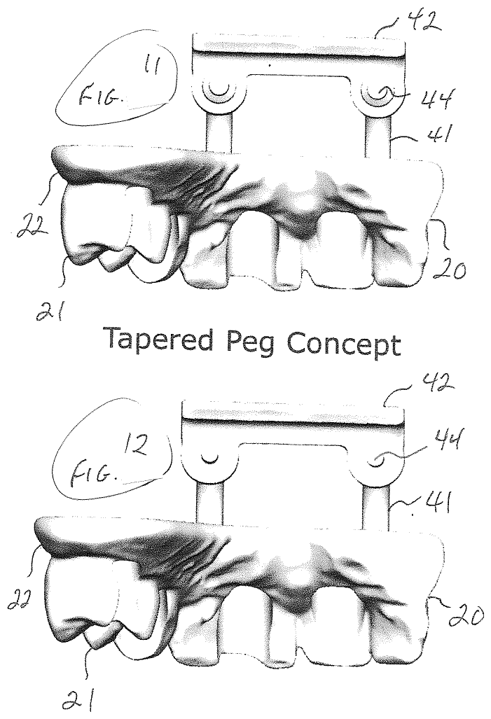

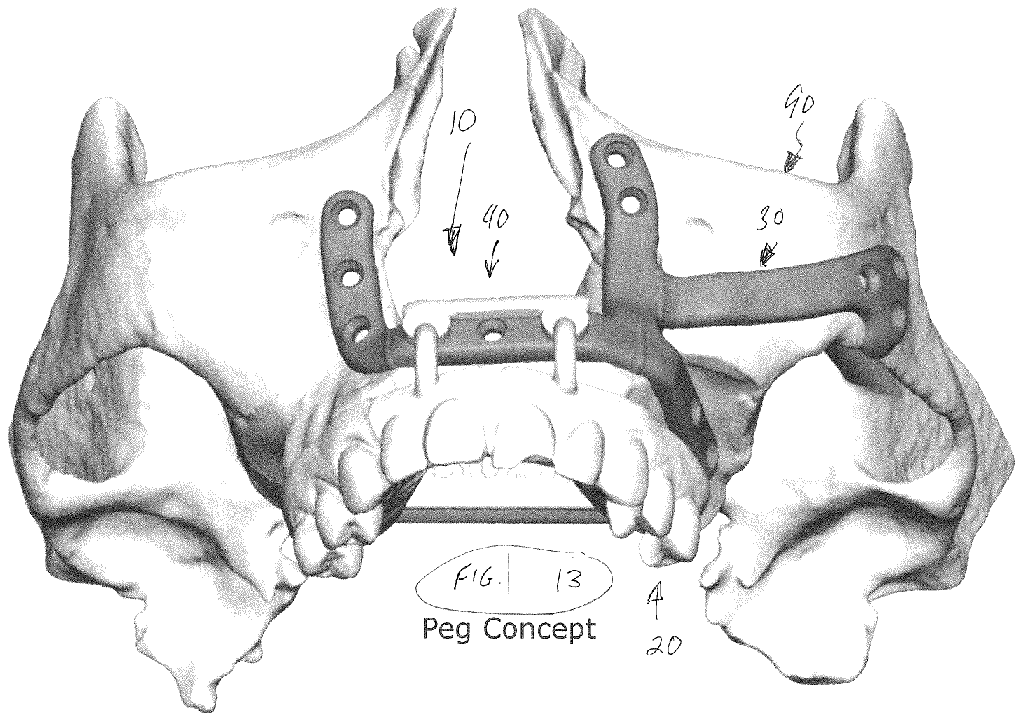

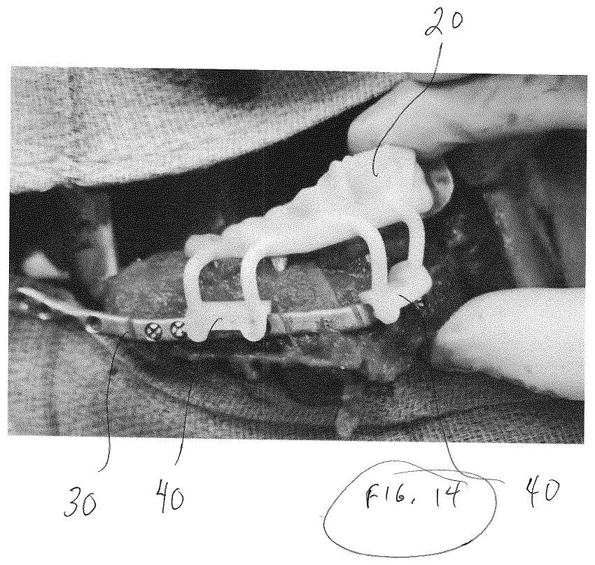

The invention in various embodiments is a process or method for designing and producing a reconstruction assembly adapted for implantation onto a maxilla or mandible to replace a resected or damaged portion, as well as the reconstruction assembly itself. FIGS. 1 through 13 illustrate the invention with relation to the reconstruction of a maxilla and will be used to provide the detailed description, but it is to be understood the invention is also applicable to reconstruction of a mandible, as shown in FIG. 14.

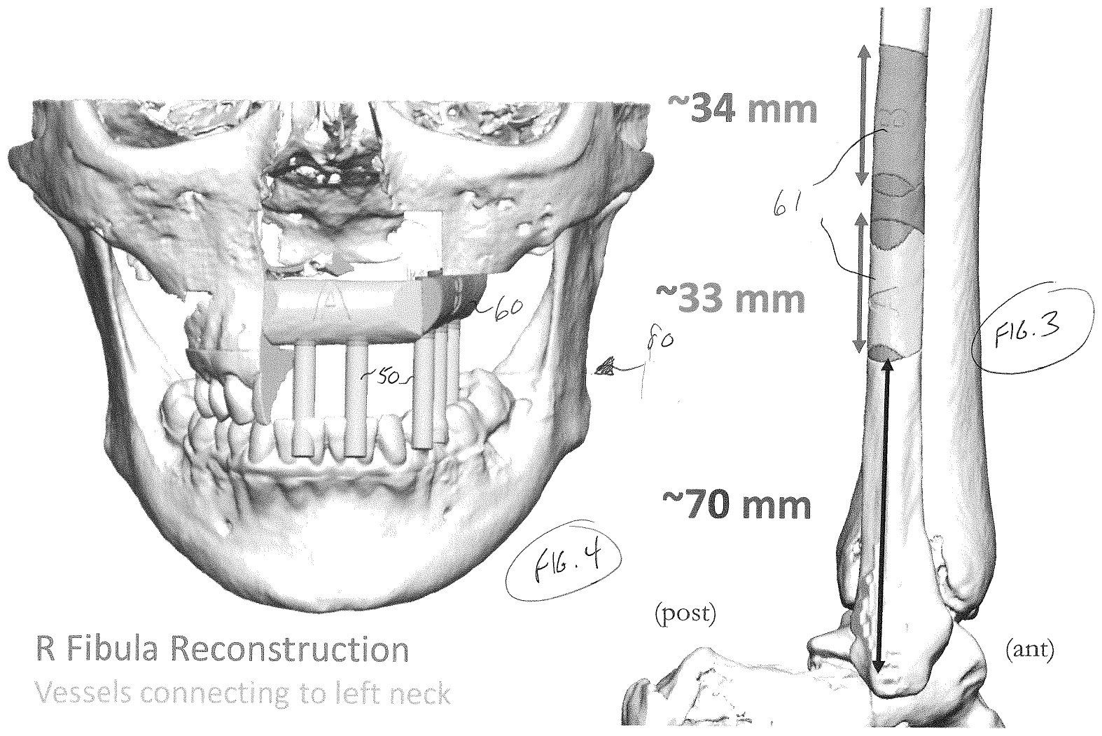

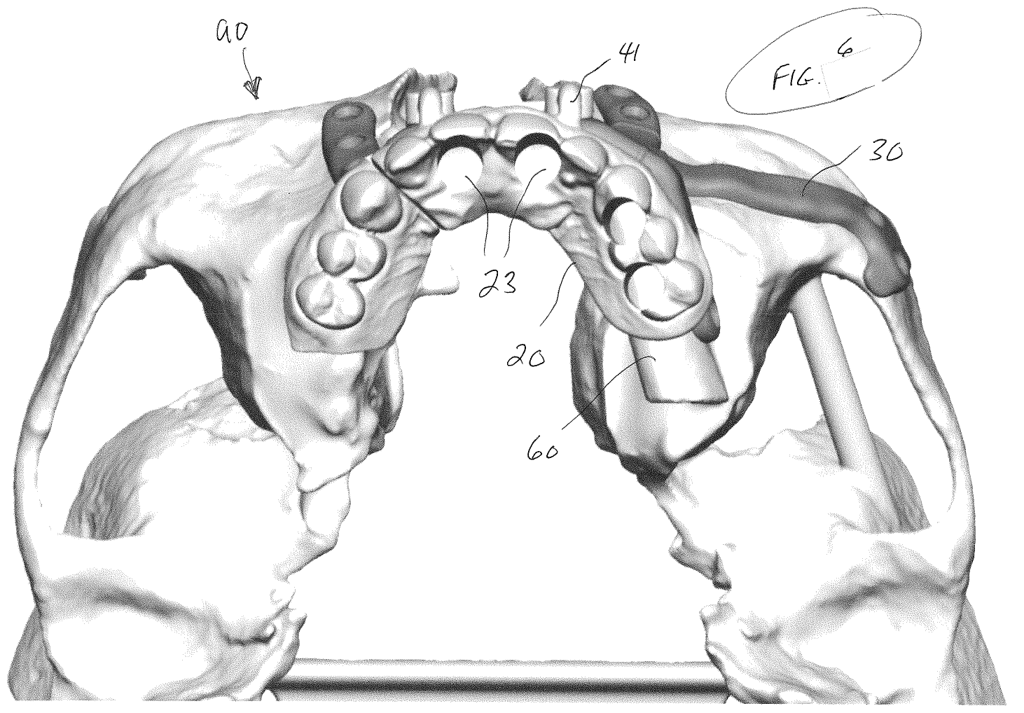

FIGS. 1 and 2 illustrate a scanned 3-D image or virtual model 80 of a patient's maxilla showing a region 81 to be resected, which will be replaced with a reconstruction assembly 10, the planned location of the fibula flap 60 and implant posts 50 being shown in FIGS. 4 and 5.

A physical 3-D model 90 of a patient's maxilla or mandible that has been resected is also created based on the imaging of maxilla or mandible of the actual patient. A virtual reconstruction assembly is now designed based on the 3-D virtual model and then an actual reconstruction assembly 10 is manufactured. The reconstruction assembly 10 comprises a fixation implant 30 contoured to the surface configuration of the patient's remaining bone after resection, a dental prosthesis 20, a removable alignment outrigger 40 joined to the dental prosthesis 20, implant posts 50 and a fibula flap 60. The implant posts 50 are inserted into a fibula prior to harvesting fibula segments 61, with the fibula segments 61 subsequently harvested to create a fibula flap 60. The fixation implant 30 is affixed to the physical 3-D model 90 using screws and the fibula segments 61 are then affixed to the fixation implant 30 to define the fibula flap 60. The alignment outrigger 40 is seated or mounted onto the fixation implant 30 with the implant posts 50 extending into bores 23 in the dental prosthesis 20. Resin is applied to cement the dental prosthesis 20 to the implant posts 50, the alignment outrigger 40 ensuring the correct location and orientation of the dental prosthesis 20 relative to the fixation implant 30. The alignment outrigger 40 is then removed from the dental prosthesis 20 and fixation implant 30 by cutting the material of composition or releasing mechanical fasteners. The reconstruction assembly 10, now minus the alignment outrigger 40, is then transitioned from the 3-D model 90 onto the patient by securing the fixation implant 30 onto the patient's bone using screws.

The fixation implant 30 is a rigid plate, frame or similar construct that conforms to the surface configuration of the patient's non-resected bone and is provided with apertures 31 to receive bone screws or similar mechanical fixation members. The purpose of the fixation implant 30 is to secure the reconstruction assembly 10 to the patient. Once the fibula segments 61 are obtained, they are mounted to the fixation implant 30 in the proper location using bone screws or the like, thereby forming a fibula flap or arch 60 onto which the dental prosthesis 20 will be mounted.

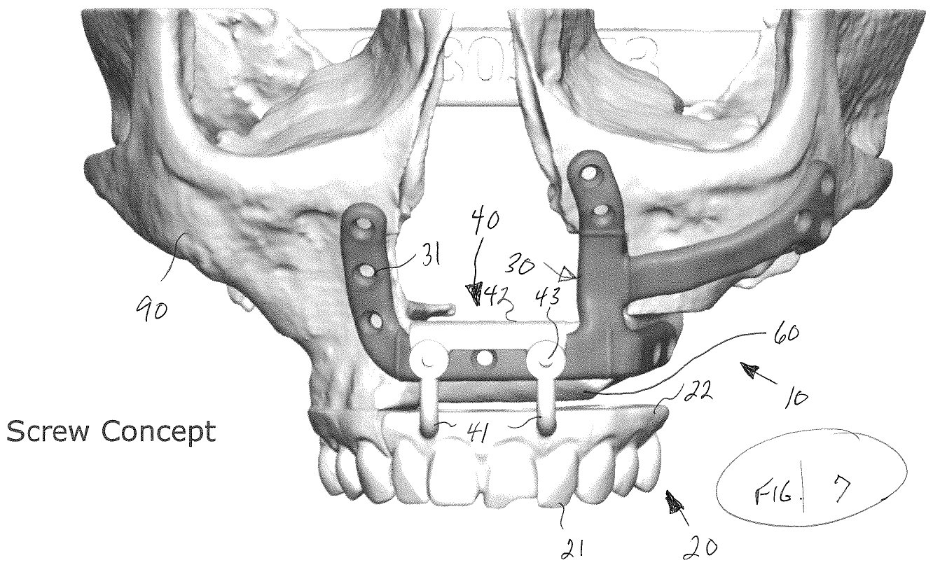

The dental prosthesis 20 is an artificial member configured with teeth 21 and tissue 22 to replace the resected elements. The dental prosthesis 20 is also provided with bores 23 to receive the implant posts 50 extending from the fibula flap 60, as shown in FIG. 6. It is important to properly locate the dental prosthesis 20 on the resected maxilla, and therefore the dental prosthesis 20 is provided with a removable alignment outrigger 40. Preferably the dental prosthesis 20 and the alignment outrigger 40 are initially manufactured as a single unit, with the material of construction of the alignment outrigger 40 being such that it can be easily severed from the dental prosthesis 20. Alternatively, the removable alignment outrigger 40 may be temporarily affixed to the dental prosthesis 20 with mechanical fasteners or similar means.

The alignment outrigger 40 comprises one or more connector arms 41 extending from the prosthesis tissue 22 to a seating saddle 42. The seating saddle 42 is configured to abut or seat on a portion of the fixation implant 30. The seating saddle 42 is temporarily connected to the fixation implant 30 by mounting means, which may for example comprise apertures 43 to receive screws (FIGS. 7-10) which pass into apertures 31 on the fixation implant 30. Alternatively, pegs 44, either cylindrical or tapered (FIGS. 11-13), may be inserted into the apertures 31 of the fixation implant 30.

With the alignment outrigger 40 mounted onto fixation implant 30, the implant posts 50 extending from the fibula flap 60 reside in the bores 23 of the dental prosthesis 20. With the dental prosthesis properly positioned, oriented and aligned, resin or other cementing material is applied into the bores 23 to secure the dental prosthesis 20 to the implant posts 50 and thereby to the fibula flap 60 and fixation implant 30 as well. Because the components have been precisely designed, the alignment outrigger 40 ensures that the dental prosthesis 20 is properly located relative to the fixation implant 30. Once the resin has cured, the arms 41 are cut or otherwise detached from the dental prosthesis 20 to release the alignment outrigger 40 from the dental prosthesis 20 such that it may now be removed from the fixation implant 30. This leaves the reconstruction assembly 10, now comprised of the fixation implant 30, the fibular flap 60, the implant posts 50 and the dental prosthesis 20, mounted onto the 3-D model 90.

The reconstruction assembly 10 is then removed from the 3-D model 90 and transferred to the patient. The fixation implant 30 is now permanently affixed to the patient's bone using bone screws or the like, thereby correctly locating the dental prosthesis 20.

It is understood that substitutions or equivalents to some steps or elements set forth above may be obvious to those of skill in the art, and therefore the true scope and definition of the invention is to be as set forth in the following claims.

We claim:

1. A jaw graft reconstruction method comprising the steps of:
(a) creating a virtual and a physical 3-D model of a patient's resected maxilla or mandible,
(b) designing and manufacturing components of a reconstruction assembly comprising a fixation implant contoured to the surface configuration of the patient's bone remaining after resection, a dental prosthesis, a removable alignment outrigger joined to the dental prosthesis, a fibula flap and implant posts,
(c) inserting the implant posts into a fibula,
(d) harvesting segments from the fibula,
(e) affixing the fixation implant to the 3-D physical model of the patient's maxilla or mandible,
(f) affixing the fibula segments to the fixation implant to create the fibula flap,
(g) mounting the alignment outrigger onto the fixation implant with the implant posts extending into bores in the dental prosthesis,
(h) applying resin to cement the dental prosthesis to the implant posts, whereby the alignment outrigger ensures the correct location and orientation of the dental prosthesis relative to the fixation implant,
(i) removing the alignment outrigger from the dental prosthesis and fixation implant, and
(j) transitioning the reconstruction assembly from the 3-D physical model onto the patient by securing the fixation implant onto the patient's bone.

2. A jaw reconstruction assembly comprising:
(a) a fixation implant contoured to the surface configuration of the patient's bone and adapted to be fastened thereon,
(b) a dental prosthesis having bores,
(c) a removable alignment outrigger temporarily joined to the dental prosthesis and adapted to be temporarily mounted onto the fixation implant,
(d) a fibula flap affixed to the fixation implant, and
(e) implant posts affixed to the fibula flap and configured to reside within the bores of the dental prosthesis, the implant posts being cemented to the dental prosthesis.
